(19) United States
(12) Patent Application Publication
(10) Pub. No.: US 2024/0407795 A1
(43) Pub. Date: Dec. 12, 2024

(54) ULTRASOUND MEDICAL DEVICE

(71) Applicant: Boston Scientific Scimed, Inc., Maple Grove, MN (US)

(72) Inventors: Kyle Allen McCleary, Coon Rapids, MN (US); Luis Alonso Quiros Fonseca, San Ramon (CR)

(73) Assignee: Boston Scientific Scimed, Inc., Maple Grove, MN (US)

(21) Appl. No.: 18/735,301

(22) Filed: Jun. 6, 2024

Related U.S. Application Data
(60) Provisional application No. 63/471,610, filed on Jun. 7, 2023.

(51) Int. Cl. A61B 17/22

(52) U.S. Cl. CPC A61B 17/2202; A61B 2017/22008; A61B 2017/22025; A61B 2017/22062

(57) ABSTRACT

Example medical devices and methods for using the medical devices are disclosed. An example ultrasound medical device includes an elongate shaft having a distal end region, a housing coupled to the elongate shaft, a first ultrasound transducer coupled to the housing, and a second ultrasound transducer coupled to the housing. Further, the first ultrasound transducer is configured to generate a first ultrasound wave, the second ultrasound transducer is configured to generate a second ultrasound wave and the first ultrasound wave constructively interferes with the second ultrasound wave to generate a localized pressure wave in order to transmit energy to a target region.

### CROSS-REFERENCE TO RELATED APPLICATIONS

This application claims the benefit of priority under 35 U.S.C. § 119 of U.S. Provisional Application No. 63/471,610, filed Jun. 7, 2023, the entire disclosure of which is hereby incorporated by reference.

### TECHNICAL FIELD

The present disclosure pertains to medical devices, and methods for manufacturing medical devices. More particularly, the present disclosure pertains to ultrasound devices and methods.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example ultrasound medical device includes an elongate shaft having a distal end region, a housing coupled to the elongate shaft, a first ultrasound transducer coupled to the housing, and a second ultrasound transducer coupled to the housing. Further, the first ultrasound transducer is configured to generate a first ultrasound wave, the second ultrasound transducer is configured to generate a second ultrasound wave and the first ultrasound wave constructively interferes with the second ultrasound wave to generate a localized pressure wave in order to transmit energy to a target region.

Alternatively or additionally to any of the embodiments above, wherein the first ultrasound transducer and the second ultrasound transducer are positioned within the housing.

Alternatively or additionally to any of the embodiments above, wherein the housing includes a first housing member and a second housing member, and wherein the first housing member is configured to attach to the second housing member in a clamshell configuration.

Alternatively or additionally to any of the embodiments above, wherein the housing member includes a first aperture, and wherein the aperture is positioned between the first ultrasound transducer and the second ultrasound transducer.

Alternatively or additionally to any of the embodiments above, wherein the transmitted energy is configured to pass through the aperture.

Alternatively or additionally to any of the embodiments above, further comprising a third ultrasound transducer and a fourth ultrasound transducer, and wherein the first ultrasound transducer and the second ultrasound transducer form a first pair of ultrasound transducers, wherein the third ultrasound transducer and the fourth ultrasound transducer form a second pair of ultrasound transducers.

Alternatively or additionally to any of the embodiments above, wherein the first pair of ultrasound transducers are longitudinally aligned with the second pair of ultrasound transducers.

Alternatively or additionally to any of the embodiments above, further comprising a balloon coupled to the elongate shaft, and wherein the balloon is designed to shift between a first unexpanded configuration and an expanded configuration wherein the balloon is in contact with the target region, and wherein the housing is positioned underneath the balloon.

Alternatively or additionally to any of the embodiments above, wherein the balloon is configured to be expanded by an inflation media, and wherein the generation of the pressure wave further includes the generation of a bubble within the inflation media.

Alternatively or additionally to any of the embodiments above, wherein the pressure wave is generated within the balloon.

Alternatively or additionally to any of the embodiments above, further comprising a third ultrasound transducer and a fourth ultrasound transducer positioned in the housing, and wherein the first ultrasound transducer and the second ultrasound transducer form a first pair of ultrasound transducers, wherein the third ultrasound transducer and the fourth ultrasound transducer form a second pair of ultrasound transducers.

Alternatively or additionally to any of the embodiments above, wherein the first pair of ultrasound transducers are configured to generate a first pressure wave within the balloon, and wherein the second pair of transducers are configured to generate a second pressure wave within the balloon, and wherein the generation of the first pressure wave occurs substantially simultaneously with generation of the second pressure wave.

Alternatively or additionally to any of the embodiments above, wherein the first pair of ultrasound transducers are configured to generate a first cavitation event within the balloon, and wherein the second pair of transducers are configured to generate a second cavitation event within the balloon, and wherein the first cavitation event occurs substantially simultaneously with the second cavitation event.

Alternatively or additionally to any of the embodiments above, wherein the first ultrasound transducer, the second ultrasound transducer or both the first and the second ultrasound transducers are designed to transmit an ultrasound wavelength at a first frequency, and wherein the first ultrasound transducer is longitudinally spaced away from the second ultrasound transducer between a distance of 0.5 to 3.5 times the first frequency wavelength.

An example ultrasound system includes a generator, an elongate shaft having a distal end region and a proximal region coupled to the generator, a balloon coupled to the distal end region of the elongate shaft and a set of ultrasound transducers coupled to the generator and positioned under the balloon. Further, the set of ultrasound transducers are configured to generate a first ultrasound wave and a second ultrasound wave, and wherein the first ultrasound wave constructively interferes with the second ultrasound wave to generate a localized cavitation event within the balloon in order to transmit energy to a target region.

Alternatively or additionally to any of the embodiments above, wherein the cavitation event further includes the generation of a pressure wave within the balloon.

An example method for treating a blood vessel includes disposing a medical device within the blood vessel at a position adjacent to a lesion. The medical device includes an elongate shaft having a distal end region, a balloon coupled to the distal end region, a first ultrasound transducer coupled to the shaft and positioned under the balloon and a second ultrasound transducer coupled to the shaft and positioned under the balloon. The method further includes generating a first ultrasound wave with the first ultrasound transducer and generating a second ultrasound wave with the second ultrasound transducer, wherein the first ultrasound wave constructively interferes with the second ultrasound wave to generate a force to at least partially break apart the lesion.

Alternatively or additionally to any of the embodiments above, wherein the balloon is designed to shift between a first unexpanded configuration and an expanded configuration wherein the balloon is in contact with the lesion.

Alternatively or additionally to any of the embodiments above, wherein the balloon is configured to be expanded by an inflation media, and wherein the generation of the force further includes the generation of a bubble within the inflation media.

Alternatively or additionally to any of the embodiments above, wherein the generation of the force further includes the generation of a pressure wave within the balloon.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an example medical device;

FIG. 2 is an exploded view of a portion of the medical device of FIG. 1;

FIG. 3 is an alternative example of a portion of the medical device of FIG. 1;

FIG. 4 is a cross-sectional view of a portion of the medical device of FIG. 1;

FIG. 5 is a cross-sectional view of an example medical device;

FIG. 6 illustrates an example medical device;

FIG. 7 is an exploded view of a portion of the medical device of FIG. 6;

FIG. 8 is a cross-sectional view of a portion of the medical device of FIG. 6;

FIG. 9 illustrates portion of an example medical device;

FIG. 10 is a partial cross-sectional view of an example medical device;

FIG. 11 is a partial cross-sectional view of an example medical device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

The use of ultrasound medical devices may be a desirable method for treating intravascular lesions in a blood vessel. In some instances, calcification along or otherwise adjacent to the vessel wall can complicate an intervention. Disclosed herein are ultrasound medical devices and methods that are designed to improve the treatment of intravascular lesions. The devices and methods disclosed herein may be described as ultrasound medical devices/methods in that the devices may transfer a force to the treatment area to break up the lesion. Some additional details are disclosed herein.

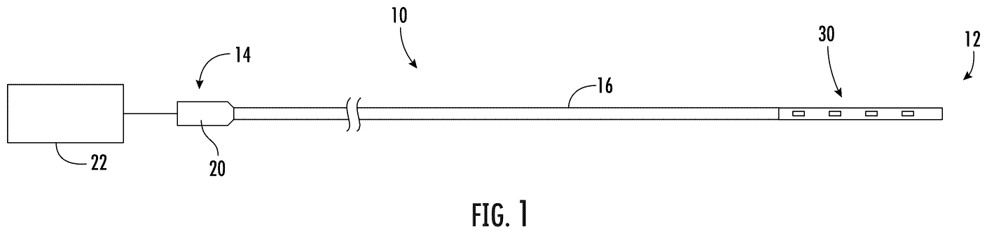

FIG. 1 illustrates an example medical device 10 having a distal end region 12 and a proximal end region 14. The medical device 10 may include a catheter shaft 16. The catheter shaft 16 may include one or more lumens extending therein. For example, the catheter shaft 16 may include a guidewire lumen. Additionally, a force transmitting assembly 30 may be coupled to the distal end region 12 of the catheter shaft 16. Other constructions are contemplated.

FIG. 1 further illustrates that the medical device 10 may further include a handle 20 coupled to a proximal end region 14 of the catheter shaft 16. The handle 20 may include one or more actuators (e.g., buttons, selectors, etc.) which permit a clinician to operate the force-transmitting members of the medical device 10. Additionally, FIG. 1 illustrates that the handle 20 may be connected to a generator 22. As will be described in greater detail below, the generator 22 may be configured to send electrical signals along electrical wires disposed within the catheter shaft 16 to force transmitting members positioned within the force transmitting assembly 30. For example, the generator 22 may be configured to send electrical signals to one or more pairs of ultrasound transducers positioned within the force transmitting assembly 30. The ultrasound transducers may convert the electrical energy received from the generator 22 to mechanical pressure waves which act upon a target site adjacent to the force transmitting assembly 30.

In at least some instances, the medical device 10 may be considered an ultrasound medical device, a thrombectomy device, an ultrasound thrombectomy device, a lithotripsy medical device, an ultrasound lithotripsy medical device and/or a lithotripsy angioplasty medical device. For the purposes of this disclosure, an ultrasound medical device may be understood to be a device designed to transfer forces to a target region in a manner that may break up the target region. In at least some instances, the transfer of forces may occur in a repeated manner with waves or flurries of force that are meant to impact the target region. The repeated force transfers could occur in a regular manner with equally spaced time intervals between transfers, or the force transfers could occur with differing time intervals between transfers. As discussed herein, the medical device 10 (as well as other medical devices disclosed herein) may use ultrasound and/or other force generators to transfer force to the target site.

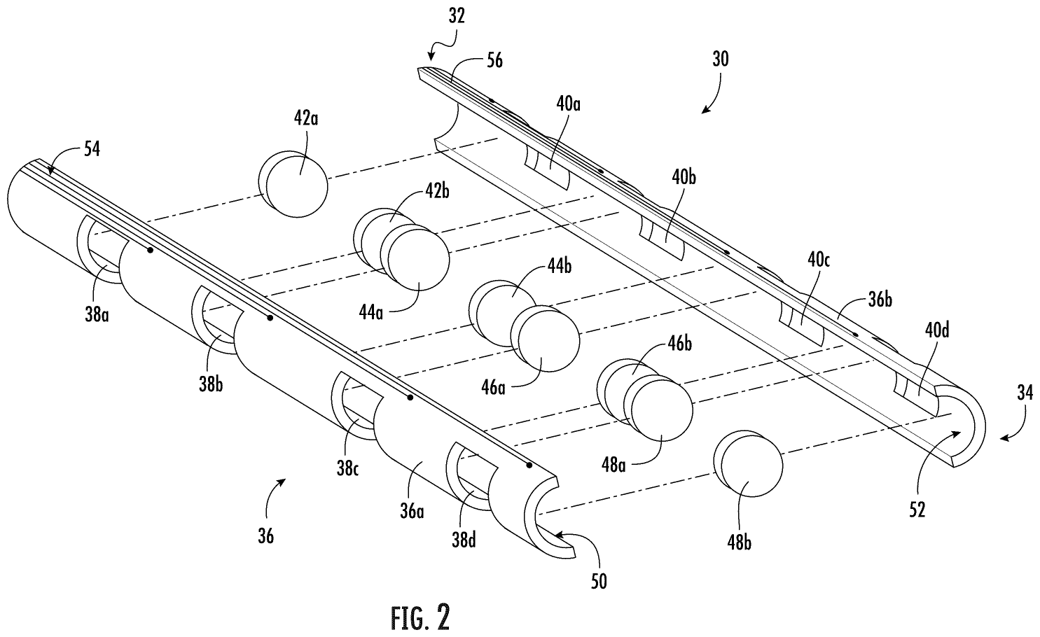

FIG. 2 illustrates an exploded view of the force transmitting assembly 30. The force transmitting assembly 30 may include a housing 36. The housing 36 may include a proximal end 32 and a distal end 34. The housing 36 may include a first housing member 36a and a second housing member 36b. The first housing member 36a and the second housing member 36b may attach to one another in a clamshell configuration. In other words, the first housing member 36a and the second housing member 36b may define two separate housing halves that, when attached to one another, define the housing 36.

FIG. 2 further illustrates that the force transmitting assembly 30 may further include one or more ultrasound transducers 42a, 42b, 44a, 44b, 46a, 46b, 48a, 48b organized into one or more pairs of ultrasound transducers 42a, 42b, 44a, 44b, 46a, 46b, 48a, 48b. In this example, the one or more pairs of ultrasound transducers 42a, 42b, 44a, 44b, 46a, 46b, 48a, 48b may be coupled to an inner surface 50, 52 of the first housing member 36a and the second housing member 36b, respectively. For example, it can be appreciated that the one or more pairs of ultrasound transducers 42a, 42b, 44a, 44b, 46a, 46b, 48a, 48b may be fixedly attached to the inner surface 50, 52 of the first housing member 36a and the second housing member 36b, respectively, prior to the first housing member 36a being attached to the second housing member 36b.

Further, in some examples the inner surface 50 of the first housing member 36a and/or the inner surface 52 of the second housing member 36b may include one or more molded portions which is designed to accept and/or secure the ultrasound transducers 42a, 42b, 44a, 44b, 46a, 46b, 48a, 48b to the inner surface 50, 52. It can be appreciated that after the ultrasound transducers 42a, 42b, 44a, 44b, 46a, 46b, 48a, 48b are positioned along an inner surface 50, 52 of the first housing member 36a and/or the second housing member 36b, the first housing member 36a may be attached to the second housing member 36b, whereby the first housing member 36a and the second housing member 36b, together, may substantially surround and secure the ultrasound transducers 42a, 42b, 44a, 44b, 46a, 46b, 48a, 48b within an interior cavity of the housing 36.

It can be appreciated from FIG. 2 that the ultrasound transducers 42a, 42b, 44a, 44b, 46a, 46b, 48a, 48b may be arranged into one or more pairs of ultrasound transducers, whereby the pairs of ultrasound transducers ultrasound transducers 42a, 42b, 44a, 44b, 46a, 46b, 48a, 48b are aligned along the longitudinal axis of the housing 30. For example, FIG. 2 illustrates that the medical device 10 may include a first pair of ultrasound transducers 42a, 42b, a second pair of ultrasound transducers 44a, 44b, a third pair of ultrasound transducers 46a, 46b and a fourth pair of ultrasound transducers 48a, 48b. In general, the number, spacing, arrangement, and configuration of the ultrasound transducers 42a, 42b, 44a, 44b, 46a, 46b, 48a, 48b can vary. For example, the medical device 10 may include one, two, three, four, five, six, seven, eight, nine, ten or more ultrasound transducers. The spacing between the ultrasound transducers 42a, 42b, 44a, 44b, 46a, 46b, 48a, 48b may be regular/even or uneven.

Further, the ultrasound transducers which define a pair of ultrasound transducers, respectively, my face one another within the housing 36. For example, the ultrasound transducer 42a may face the ultrasound transducer 42b, the ultrasound transducer 44a may face the ultrasound transducer 44b, the ultrasound transducer 46a may face the ultrasound transducer 46b and the ultrasound transducer 48a may face the ultrasound transducer 48b.

FIG. 2 further illustrates that the medical device 10 may include one or more electrical leads 54, 56 coupled to the ultrasound transducers 42a, 42b, 44a, 44b, 46a, 46b, 48a, 48b. The electrical leads 54, 56 may extend to the power supply or generator 22 shown in FIG. 1. In some examples, an individual, dedicated electrical lead may extend to each ultrasound transducer 42a, 42b, 44a, 44b, 46a, 46b, 48a, 48b. For example, FIG. 2 illustrates four electrical leads 56 which may extend from the generator 22 to the ultrasound transducers 42a, 44a, 46a, 48a. Likewise, FIG. 2 illustrates four electrical leads 54 which may extend from the generator 22 to the ultrasound transducers 42b, 44b, 46b, 48b.

However, in other examples, a single electrical lead may be utilized to power both ultrasound transducers which define a pair of ultrasound transducers 42a, 42b, 44a, 44b, 46a, 46b, 48a, 48b. For example, a single electrical lead may be utilized to power the first pair of ultrasound transducers 42a, 42b, another electrical lead may be utilized to power the second pair of ultrasound transducers 44a, 44b, another electrical lead may be utilized to power the third pair of ultrasound transducers 46a, 46b and another electrical lead may be utilized to power the fourth pair of ultrasound transducers 48a, 48b.

In some examples, the electrical leads 54, 56 attached to the ultrasound transducers 42a, 42b, 44a, 44b, 46a, 46b, 48a, 48b may extend along the outer surface of the housing 36. In other examples, the electrical leads 54, 56 attached to the ultrasound transducers 42a, 42b, 44a, 44b, 46a, 46b, 48a, 48b may extend along the inner surface of the housing 36. In yet other examples, the electrical leads 54, 56 attached to the ultrasound transducers 42a, 42b, 44a, 44b, 46a, 46b, 48a, 48b may extend within (e.g., through) the wall of the housing 36.

Similarly, in some examples, the electrical leads 54, 56 attached to the ultrasound transducers 42a, 42b, 44a, 44b, 46a, 46b, 48a, 48b may extend along the outer surface of the catheter shaft 16. In other examples, the electrical leads 54, 56 attached to the ultrasound transducers 42a, 42b, 44a, 44b, 46a, 46b, 48a, 48b may extend along the inner surface of the catheter shaft 16. In yet other examples, the electrical leads 54, 56 attached to the ultrasound transducers 42a, 42b, 44a, 44b, 46a, 46b, 48a, 48b may extend within the wall of the catheter shaft 16.

FIG. 2 further illustrates that the housing member 36a may include one or more apertures 38a, 38b, 38c, 38d and the housing member 36b may include one or more apertures 40a, 40b, 40c, 40d configured to permit a force (e.g., pressure wave, cavitation bubble, etc.) to pass through the first housing member 36a and/or the second housing member 36b. It can be appreciated that a force (e.g., pressure wave, cavitation bubble, etc.) may pass from the first pair of ultrasound transducers 42a, 42b, the second pair of ultrasound transducers 44a, 44b, the third pair of ultrasound transducers 46a, 46b and/or the fourth pair of ultrasound transducers 48a, 48b through an aperture 38a, 38b, 38c, 38d of the first housing member 36a and/or the apertures 40a, 40b, 40c, 40d of the second housing member 36b. It can be appreciated that the apertures 38a, 38b, 38c, 38d, 40a, 40b, 40c, 40d may include a variety of shapes and sizes. For example, the apertures 38a, 38b, 38c, 38d, 40a, 40b, 40c, 40d may be square, rectangular, circular, ovular, triangular, polygonal, or any combinations thereof.

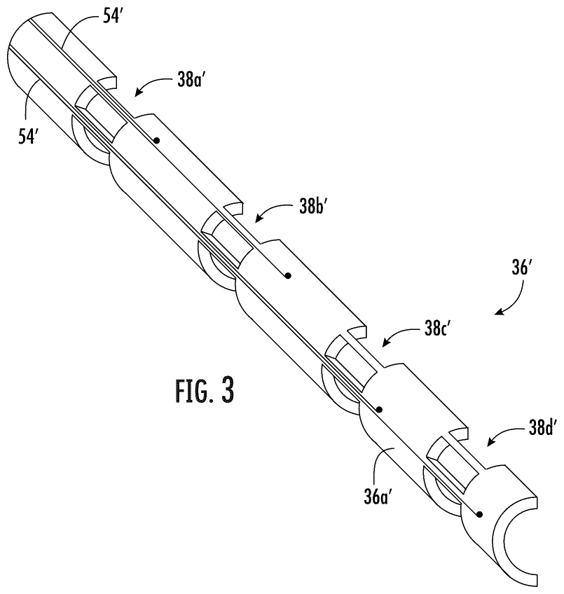

FIG. 3 illustrates another example embodiment for the housing 36a described herein with respect to FIG. 2. For example, FIG. 3 illustrates a housing member 36a′ of an example housing 36′. It can be appreciated that the housing member 36a′ may represent each mirrored half of a clamshell housing 36′, similar to the housing members 36a, 36b described with respect to the housing member 36.

FIG. 3 illustrates that the housing 36′ may include aperture regions 38a′, 38b′, 38c′, 38d′. FIG. 3 further illustrates that the aperture regions 38a′, 38b′, 38c′, 38d′ may be spaced along the longitudinal axis of the housing 36′. Further, the aperture regions 38a′, 38b′, 38c′, 38d′ may align with the ultrasound transducers 42a, 42b, 44a, 44b, 46a, 46b, 48a, 48b. Additionally, FIG. 3 illustrates that each of the aperture regions 38a′, 38b′, 38c′, 38d′ may include one or more individual apertures radially spaced around the longitudinal axis of the housing 36′.

Further, FIG. 3 further illustrates one or more electrical leads 54′, 56′ extending along the housing 36′. The electrical leads 54′, 56′ may be similar in form and function to the electrical leads 54, 56 described herein. The electrical leads 54′, 56′ may be coupled to the ultrasound transducers 42a, 42b, 44a, 44b, 46a, 46b, 48a, 48b. The electrical leads 54′, 56′ may extend to the power supply or generator 22 shown in FIG. 1.

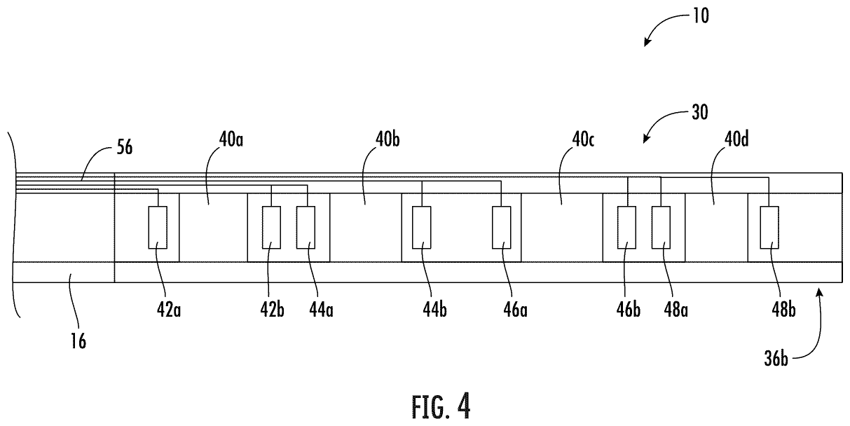

FIG. 4 illustrates a cross-sectional view of a portion of the medical device 10 of FIG. 1. FIG. 4 illustrates the force transmitting member 30 coupled to the distal end of the catheter shaft 16. Further, FIG. 4 illustrates the first pair of ultrasound transducers 42a, 42b, the second pair of ultrasound transducers 44a, 44b, the third pair of ultrasound transducers 46a, 46b and the fourth pair of ultrasound transducers 48a, 48b aligned longitudinally along the second housing member 36b.

FIG. 4 further illustrates that the first pair of ultrasound transducers 42a, 42b may be positioned on either side of the apertures 38a, 40a, the second pair of ultrasound transducers 44a, 44b may be positioned on either side of the apertures 38b, 40b, the third pair of ultrasound transducers 46a, 46b may be positioned on either side of the apertures 38c, 40c and the fourth pair of ultrasound transducers 48a, 48b may be positioned on either side of the apertures 38d, 40d. It can be further appreciated that a force (e.g., cavitation bubble, pressure wave, etc.) generated by the first pair of ultrasound transducers 42a, 42b may pass through the apertures 38a, 40a, a force (e.g., cavitation bubble, pressure wave, etc.) generated by the second pair of ultrasound transducers 44a, 44b may pass through the apertures 38b, 40b, a force (e.g., cavitation bubble, pressure wave, etc.) generated by the third pair of ultrasound transducers 46a, 46b may pass through the apertures 38c, 40c and a force (e.g., cavitation bubble, pressure wave, etc.) generated by the fourth pair of ultrasound transducers 48a, 48b may pass through the apertures 38d, 40d. FIG. 4 further illustrates the electrical leads 56 attached to one or more of the ultrasound transducers 42a, 42b, 44a, 44b, 46a, 46b, 48a, 48b. While not visible in FIG. 4, it can be appreciated that the electrical leads 54 may be attached to one or more of the ultrasound transducers 42a, 42b, 44a, 44b, 46a, 46b, 48a, 48b.

Activating the ultrasound transducers 42a, 42b, 44a, 44b, 46a, 46b, 48a, 48b may utilize constructive ultrasound wave interference to create cavitation events, whereby the cavitation events may collapse and cause pressure waves (e.g., shockwaves) to pass through the apertures 38a, 38b, 38c, 38d, 40a, 40b, 40c, 40d in the housing 36 and break up calcified lesions at a target site. For example, activating the ultrasound transducers 42a, 42b, 44a, 44b, 46a, 46b, 48a, 48b with electrical energy provided by the generator 22 may create constructive ultrasound wave interference to create cavitation events, whereby the cavitation events may collapse and cause pressure waves (e.g., shockwaves) to pass through apertures 38a, 38b, 38c, 38d, 40a, 40b, 40c, 40d in the housing 36 and break up calcified lesions. The cavitation events may be produced with or without a nucleation aid.

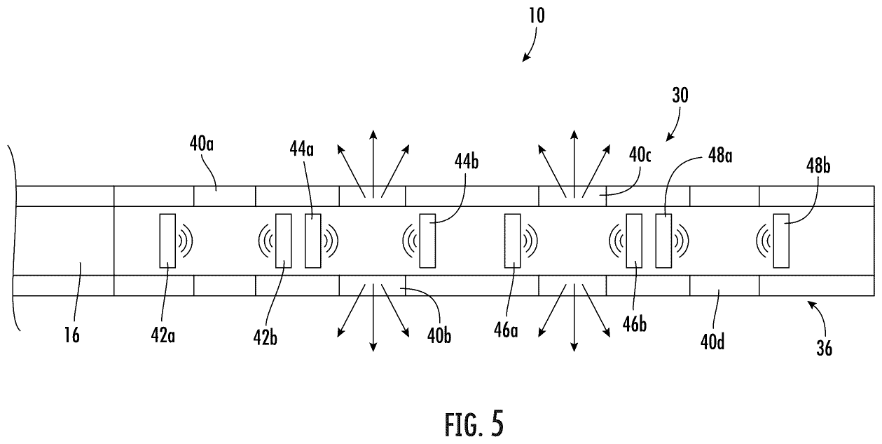

FIG. 5 illustrates a top cross-sectional view of the second pair of ultrasound transducers 44a, 44b and the third pair of ultrasound transducers 46a, 46b utilizing constructive ultrasound wave interference to create cavitation events and resultant pressure waves (e.g., shockwaves) that pass through the apertures 38b, 40b, 38c, 40c in the housing 36. The resultant pressure waves are shown (via the arrows in FIG. 5) passing into and through the apertures 40b, 40c in the housing 36. It can be appreciated that to create the cavitation event and pressure waves shown in FIG. 5, the ultrasound transducer of the 44a and the ultrasound transducer 44b may each receive electrical energy from the generator 22, whereby the ultrasound transducer of the 44a converts that electrical energy into an ultrasound wave which travels toward the ultrasound transducer 44b. Similarly, the ultrasound transducer 44b may also convert electrical energy into an ultrasound wave which travels toward the ultrasound transducer 44a. It can be appreciated that the ultrasound wave generated by the ultrasound transducer 44a may engage (e.g., collide, interfere, etc.) with the ultrasound wave generated by the ultrasound transducer 44b. It can be appreciated the ultrasound waves generated by the ultrasound transducer 44a and the ultrasound transducer 44b may constructively interference with one another to create a cavitation event and resultant pressure wave (e.g., shockwaves) that passes through the aperture 40b and to a target site (e.g., calcified lesion).

In a similar manner as that described above with respect to the second pair of ultrasound transducers 44a, 44b, FIG. 5 further illustrates another cavitation event and resultant pressure wave being generated by the third pair of ultrasound transducers 46a, 46b and passing through the aperture 40c and to a target site (e.g., calcified lesion).

It can be further appreciated that cavitation events and resultant pressure waves may also be generated by the first pair of ultrasound transducers 42a, 42b and the fourth pair of ultrasound transducers 48a, 48b. Further, it can be appreciated that the medical device 10 may be configured to generate cavitation events and resultant pressure waves in a variety of sequences. For example, the medical device 10 may be configured to create cavitation events and resultant pressure waves with all four pairs of ultrasound transducers 42a, 42b, 44a, 44b, 46a, 46b, 48a, 48b simultaneously.

Additionally, the medical device 10 may be configured to create cavitation events and resultant pressure waves sequentially between the four pairs of ultrasound transducers 42a, 42b, 44a, 44b, 46a, 46b, 48a, 48b. For example, the medical device 10 may be configured to generate sequential cavitation events and resultant pressure waves starting from the first pair of transducers 42a, 42b, to the second pair of transducers 44a, 44b, to the third pair of transducers 46a, 46b, to the fourth pair of transducers 48a, 48b. Similarly, the medical device 10 may be configured to generate cavitation events and resultant pressure waves starting from the fourth pair of transducers 48a, 48b, to the third pair of transducers 46a, 46b, to the second pair of transducers 44a, 44b, to the fourth pair of transducers 42a, 42b. It can be appreciated that the medical device 10 may be configured to generate cavitation events and resultant pressure waves in any combination and/or order among the pairs of ultrasound transducers 42a, 42b, 44a, 44b, 46a, 46b, 48a, 48b.

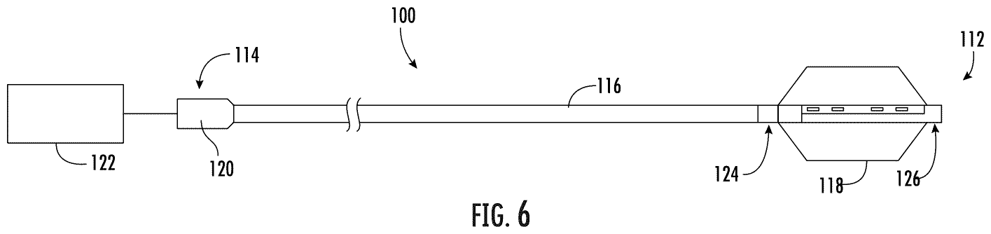

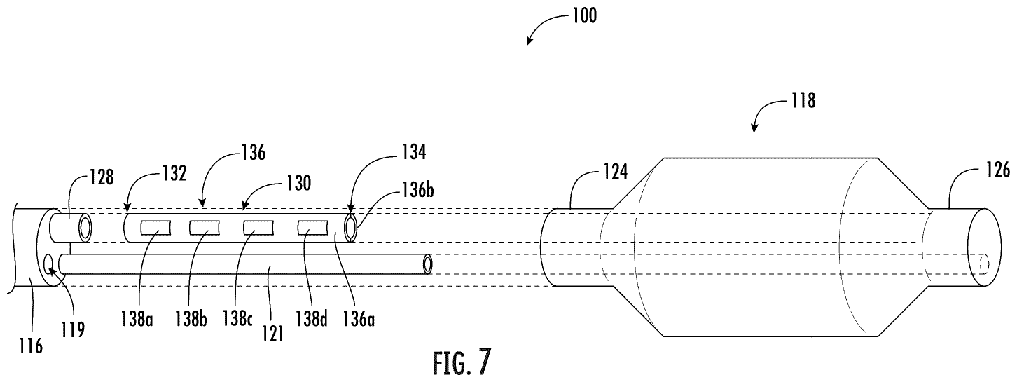

FIG. 6 illustrates another example medical device 100 having a distal end region 112 and a proximal end region 114. The medical device 100 may be similar in form and function to the medical device 10 described above. For example, the medical device 100 may include a catheter shaft 116. The catheter shaft 116 may include one or more lumens extending therein. For example, the catheter shaft 116 may include a guidewire lumen 121 (shown in FIG. 7). In some embodiments (such as those discussed above), the medical device may be free of and/or may not include a balloon, or any structure, surrounding or more or all of the ultrasound transducers. In some embodiments, the entire device is free of a balloon. However, in other embodiments (such as that shown in FIG. 6), a balloon 118 may be coupled to the distal end region 112 of the catheter shaft 116. In some instances, the balloon 118 may include a proximal waist 124, a distal waist 126 and a body region extending therebetween. The proximal waist 124 may be coupled to a portion of the distal end of the catheter shaft 116.

Further, FIG. 6 illustrates that the medical device 100 further include a force transmitting assembly 130 positioned underneath the balloon 118. The force transmitting assembly 130 may be similar in form and function to the force transmitting assembly 30 described herein. For example, the force transmitting assembly 130 may include a housing 130 formed from a first housing member 136a and a second housing member 136b. The first housing member 136a and the second housing member 136 may be similar in form and function to the first housing member 36a and the second housing member 36b described herein.

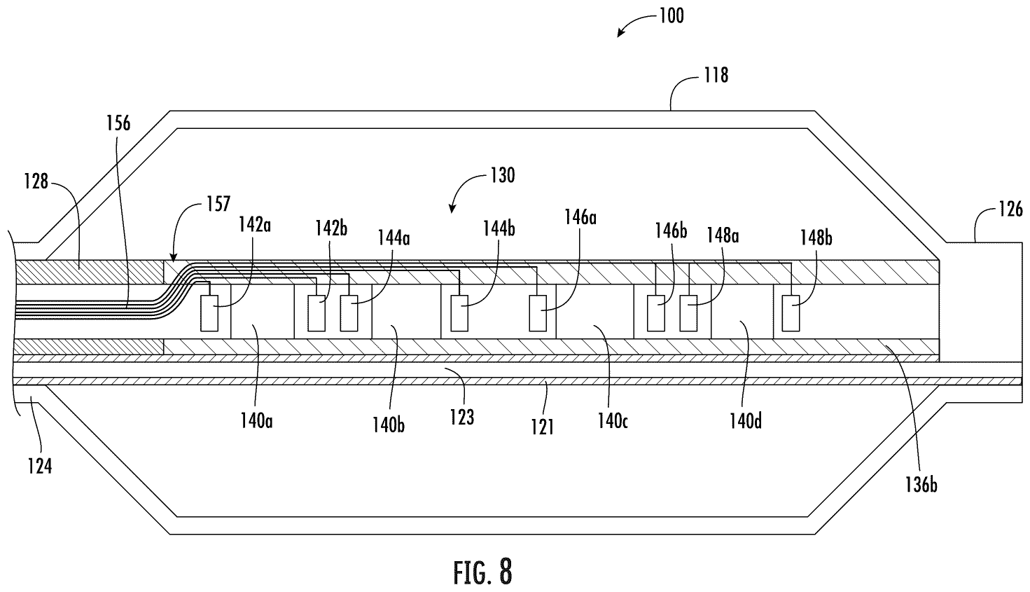

Further, the force transmitting assembly 130 may include ultrasound transducers 142a, 142b, 144a, 144b, 146a, 146b, 148a, 148b (shown in FIG. 8). The ultrasound transducers 142a, 142b, 144a, 144b, 146a, 146b, 148a, 148b may be similar in form and function to the ultrasound transducers 42a, 42b, 44a, 44b, 46a, 46b, 48a, 48b described herein. Further, the housing member 136a may include apertures 138a, 138b, 138c, 138d (shown in FIG. 7) and the housing member 138b may include apertures 140a, 140b, 140c, 140d (shown in FIG. 8) which are similar in form and function to the apertures 38a, 38b, 38c, 38d, 40a, 40b, 40c, 40d described herein. Other constructions are contemplated.

FIG. 6 further illustrates that the medical device 100 may further include a handle 120 coupled to a proximal end of the catheter shaft 116. The handle 120 may include one or more actuators (e.g., buttons, selectors, etc.) which permit a clinician to operate the force-transmitting members of the medical device 100. Additionally, FIG. 6 illustrates that the handle 120 may be connected to a generator 122. As will be described in greater detail below, the generator 122 may be configured to send electrical signals along electrical wires disposed within the catheter shaft 116 to force transmitting members positioned within the balloon 118. For example, the generator 122 may be configured to send electrical signals to one or more pairs of ultrasound transducers positioned underneath the balloon 118. The ultrasound transducers may convert the electrical energy received from the generator 122 to mechanical pressure waves which pass through balloon inflation media and act upon a target site adjacent to the balloon 118.

FIG. 7 is an exploded view of the distal end region 112 of the medical device 100. FIG. 7 illustrates that the proximal waist 124 of the balloon 118 may be attached to a portion of the distal end region of the catheter shaft 116. Further, FIG. 7 illustrates that the medical device 100 further include a force transmitting assembly 130 positioned within (e.g., underneath) the balloon 118. As will be discussed in greater detail herein, the force transmitting assembly 130 may include one or more ultrasound transducers positioned within a housing 136. As illustrated in FIG. 7, the housing 136 may include a proximal end 132 and a distal end 134. The proximal end 132 of the housing 136 may be attached to a projection 128 of the catheter shaft 116. The projection 128 may be a portion of the catheter shaft 116 which extends distally from the catheter shaft 116 and is designed to attach to the proximal end 132 of the housing 136. In other words, the outer diameter of the projection 128 may match the outer diameter of the housing 136. Further, the distal end 134 of the housing 136 may be attached to a portion of the balloon 118.

FIG. 7 illustrates that the medical device 100 may also include a guidewire shaft 121. The guidewire shaft 121 may be a portion of the catheter shaft 116 which extends distally from the catheter shaft 116 and is designed to attach to the distal waist 126 of the balloon 118. Additionally, FIG. 7 illustrates that the catheter shaft 116 may further include an inflation lumen 119. The inflation lumen 119 may be designed to permit balloon inflation media to pass from a position outside the body into the balloon 118.

In at least some instances, the medical device 100 may be considered an ultrasound medical device, a thrombectomy device, an ultrasound thrombectomy device, a lithotripsy medical device, an ultrasound lithotripsy medical device and/or a lithotripsy angioplasty medical device. For the purposes of this disclosure, an ultrasound medical device may be understood to be a device designed to transfer forces to a target region in a manner that may break up the target region. In at least some instances, the transfer of forces may occur in a repeated manner with waves or flurries of force that are meant to impact the target region. The repeated force transfers could occur in a regular manner with equally spaced time intervals between transfers, or the force transfers could occur with differing time intervals between transfers. As discussed herein, the medical device 100 (as well as other medical devices disclosed herein) may use ultrasound and/or other force generators to transfer force to the target site.

FIG. 8 illustrates a partial cross-sectional view of the medical device 100 of FIG. 6. FIG. 8 illustrates the force transmitting member 130 positioned within the balloon 118, whereby the proximal end of the force transmitting member 130 is attached to the distal end of the projection 128 of the catheter shaft 116. Additionally, FIG. 8 illustrates a guidewire lumen 123 of the guidewire shaft 121 extending from the catheter shaft 116 to the distal waist 126 of the balloon 118.

FIG. 8 further illustrates the second housing member 136b (housing member 136a is not visible in FIG. 8) positioned within the balloon 118. Further, FIG. 8 illustrates the first pair of ultrasound transducers 142a, 142b, the second pair of ultrasound transducers 144a, 144b, the third pair of ultrasound transducers 146a, 146b and the fourth pair of ultrasound transducers 148a, 148b aligned longitudinally along the second housing member 136b.

FIG. 8 further illustrates that the first pair of ultrasound transducers 42a, 42b may be positioned on either side of the apertures 138a, 140a, the second pair of ultrasound transducers 144a, 144b may be positioned on either side of the apertures 138b, 140b, the third pair of ultrasound transducers 146a, 146b may be positioned on either side of the apertures 138c, 140c and the fourth pair of ultrasound transducers 148a, 148b may be positioned on either side of the apertures 138d, 140d.

It can be further appreciated that a force (e.g., cavitation bubble, pressure wave, etc.) generated by the first pair of ultrasound transducers 142a, 142b may pass through the apertures 138a, 140a, a force (e.g., cavitation bubble, pressure wave, etc.) generated by the second pair of ultrasound transducers 144a, 144b may pass through the apertures 138b, 140b, a force (e.g., cavitation bubble, pressure wave, etc.) generated by the third pair of ultrasound transducers 146a, 146b may pass through the apertures 138c, 140c and a force (e.g., cavitation bubble, pressure wave, etc.) generated by the fourth pair of ultrasound transducers 148a, 148b may pass through the apertures 138d, 140d.

FIG. 8 further illustrates the electrical leads 156 attached to one or more of the ultrasound transducers 142a, 142b, 144a, 144b, 146a, 146b, 148a, 148b may pass through an aperture 157 in the housing member 136 and through a lumen of the projection 128. The electrical leads 156 may further pass through a lumen of the catheter shaft 116 which is aligned with the lumen of the projection 128 to the handle 120 and/or generator 122.

Activating the ultrasound transducers 142a, 142b, 144a, 144b, 146a, 146b, 148a, 148b may utilize constructive ultrasound wave interference to create cavitation events, whereby the cavitation events may collapse and cause pressure waves (e.g., shockwaves) to pass through the balloon inflation media and break up calcified lesions. For example, activating the ultrasound transducers 142a, 142b, 144a, 144b, 146a, 146b, 148a, 148b with electrical energy provided by the generator 122 may create constructive ultrasound wave interference to create cavitation events, whereby the cavitation events may collapse and cause pressure waves (e.g., shockwaves) to pass through the balloon inflation media and break up calcified lesions.

It can be appreciated that the medical device 100 may be advanced through a blood vessel to a position adjacent to the calcified lesion. When suitably positioned, the balloon 118 may be inflated as schematically depicted in FIG. 6. Inflating the balloon 118 may occur by infusing an inflation media into the balloon 118 (e.g., via an inflation lumen) that may be defined within the catheter shaft 116. Inflating the balloon 118 may include inflating the balloon 118 so that the balloon 118 comes into contact with the wall of a blood vessel. This may include simply contacting the vessel wall or, in some instances, inflating the balloon 18 may include expanding the blood vessel.

With the balloon 118 inflated (or partially inflated), the first pair of ultrasound transducers 142a, 142b, the second pair of ultrasound transducers 144a, 144b, the third pair of ultrasound transducers 146a, 146b and/or a fourth pair of ultrasound transducers 148a, 148b may be activated. As discussed herein, activating the first pair of ultrasound transducers 142a, 142b, the second pair of ultrasound transducers 144a, 144b, the third pair of ultrasound transducers 146a, 146b and/or a fourth pair of ultrasound transducers 148a, 148b may create a force (e.g., pressure wave, cavitation bubble, etc.) that can be transferred to a calcified lesion. The transferred forces may contact and break up the calcified lesion. With the calcified lesion broken up, the balloon 118 may be further inflated to a second pressure, greater than the first pressure, to treat the blood vessel.

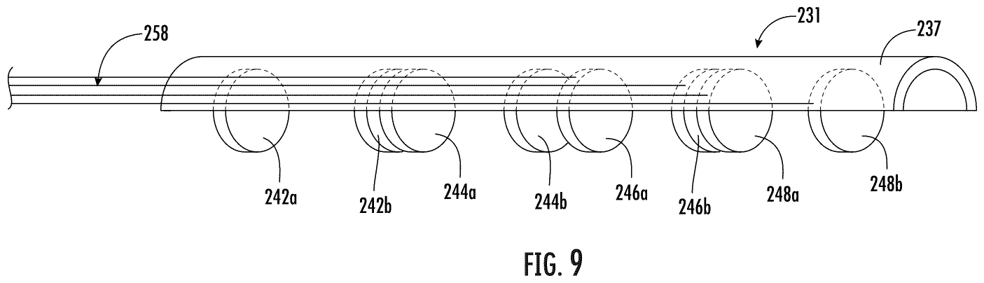

FIG. 9 illustrates another example force transmitting assembly 231 which may be utilized similarly to the force transmitting assemblies 30, 130 described herein. The force transmitting assembly 231 illustrated in FIG. 9 may include ultrasound transducers 242a, 242b, 244a, 244b, 246a, 246b, 248a, 248b attached to a spine 237 (e.g., band, strip, ribbon, etc.). The spine may be formed from a variety of materials. For example, the spine 237 may be formed from silicone or a similar material. The ultrasound transducers 242a, 242b, 244a, 244b, 246a, 246b, 248a, 248b may be similar in form and function to the ultrasound transducers 42a, 42b, 44a, 44b, 46a, 46b, 48a, 48b described herein. FIG. 8 further illustrates one or more electrical leads 258 which may be coupled to one or more of the ultrasound transducers 242a, 242b, 244a, 244b, 246a, 246b, 248a, 248b and may extend to the generator 22 shown in FIG. 1.

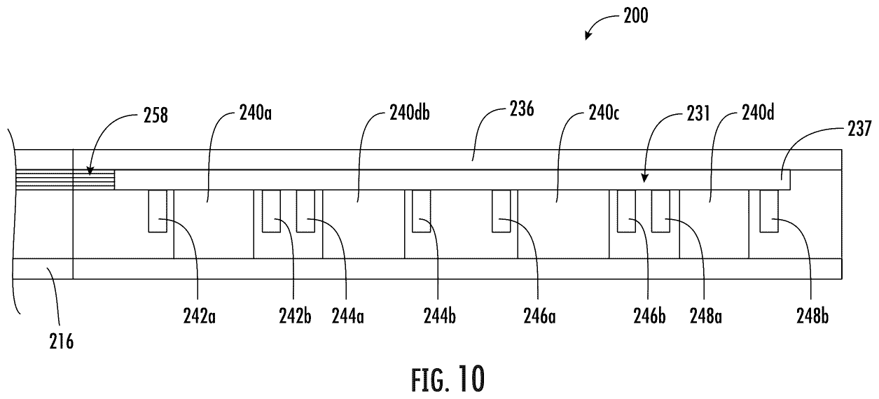

FIG. 10 illustrates a partial cross-sectional view of an example medical device 200. A force transmitting member 231 may be utilized with the medical device 200. The medical device 200 may be similar in form and function to the medical device 10 described herein. However, alternatively to the medical device 10 shown in FIG. 1, FIG. 10 illustrates the force transmitting member 231 (shown in FIG. 9) inserted into the lumen of a housing 236. It can be appreciated that the housing 236 may be similar in form and function to the housing 36 described herein. The spine 237 may be coupled to an inner surface of the housing 236.

Further, FIG. 10 illustrates the first pair of ultrasound transducers 242a, 242b positioned on either side of aperture 240a, the second pair of ultrasound transducers 244a, 244b positioned on either side of the aperture 240b, the third pair of ultrasound transducers 246a, 246b positioned on either side of the aperture 240c and the fourth pair of ultrasound transducers 248a, 248b positioned on either side of the aperture 240d. While not visible in FIG. 10, it can be appreciated that the housing 236 may also include additional apertures which are similar in form and function to the apertures 38a, 38b, 38c, 38d described herein.

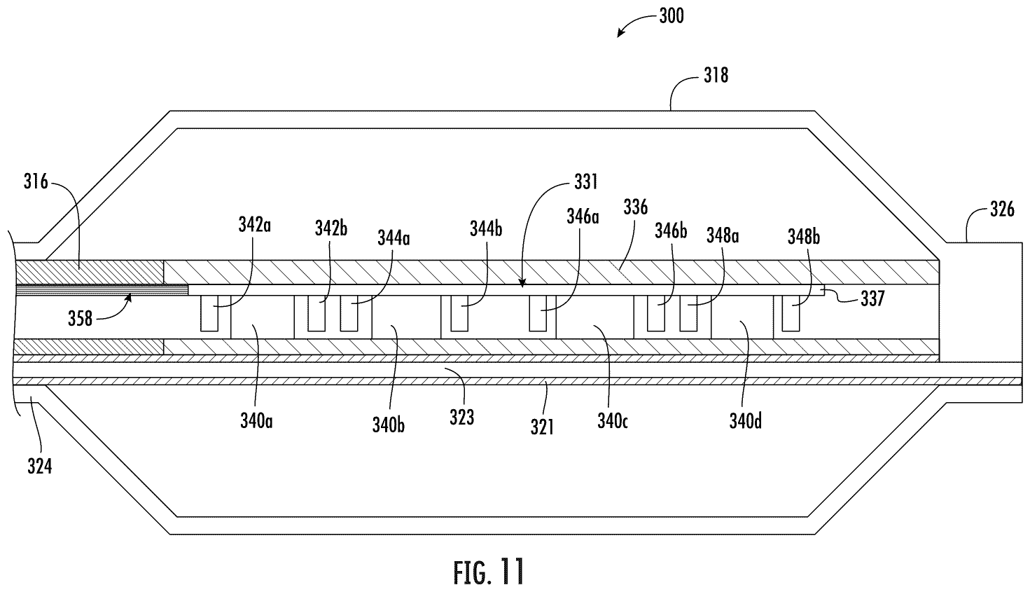

FIG. 11 illustrates a cross-sectional view of another example medical device 300. A force transmitting member 331 (similar in form and function to the force transmitting member 231 shown in FIG. 9) utilized with example medical device 300. The medical device 300 may be similar in form and function to the medical device 100 described herein. For example, FIG. 11 illustrates a guidewire lumen 323 of the guidewire shaft 321 extending from the catheter shaft 316 to the distal waist 326 of the balloon 318.

Further, FIG. 11 illustrates a housing 336 positioned within the balloon 318 of the medical device 300. However, alternatively to the medical device 100 shown in FIGS. 6-8, FIG. 11 illustrates the force transmitting member 331 has been inserted into a lumen of the housing 336. It can be appreciated that the housing 336 may be similar in form and function to the housing 136 described herein. For example, FIG. 11 illustrates the force transmitting member 331 (including the ultrasound transducers 342a, 342b, 344a, 344b, 346a, 346b, 348a, 348b attached to the spine 337) may be positioned within the lumen of the housing 336, whereby the housing 336 is further positioned underneath the balloon 318 of the medical device 300. The spine 337 may be coupled to an inner surface of the housing 336. FIG. 11 further illustrates one or more electrical leads 358 which may be coupled to one or more of the ultrasound transducers 342a, 342b, 344a, 344b, 346a, 346b, 348a, 348b and may extend to the generator 22 shown in FIG. 1.

Further, FIG. 11 illustrates the first pair of ultrasound transducers 342a, 342b positioned on either side of aperture 340a, the second pair of ultrasound transducers 344a, 344b positioned on either side of the aperture 340b, the third pair of ultrasound transducers 346a, 346b positioned on either side of the aperture 340c and the fourth pair of ultrasound transducers 348a, 348b positioned on either side of the aperture 340d. While not visible in FIG. 11, it can be appreciated that the housing 336 may also include apertures 338a, 338b, 338c, 338d which are similar in form and function to the apertures 38a, 38b, 38c, 38d described herein.

As discussed herein, it can be appreciated that the cavitation events (e.g., bubbles) and/or pressure waves may transmit mechanical energy and/or a mechanical force to a target region. In cases where the target region is a calcified lesion, the transferred force (e.g., pressure wave, cavitation bubble) may break up the calcified lesion.

It can be appreciated that for any of the example medical devices 10, 100, 200, 300 described herein, each ultrasound transducers of a given transducer pair may emit a frequency wavelength. The ultrasound transducers disclosed herein may operate within a wide range of frequency wavelengths. In some examples, the ultrasound wavelength frequency emitted by each transducer of a transducer pair may be in a range of about 1-30 MHz, or in a range of about 10-25 MHz, or in a range of about 5-15 MHz, or in a range of about 1-10 MHz, or about 15 MHz. In other examples, the ultrasound wavelength frequency emitted by each transducer of a transducer pair may be in a range of about 100 kHz to about 1000 kHz, or in a range of about 200-800 KHz, or in a range of about 400-600 MHz, or in a range of about 500 MHZ.

Further, the medical devices 10, 100, 200, 300 may be designed such that the distance between the transducers of any ultrasound transducer pair may be (N+1.5) times the wavelength emitted by the ultrasound transducers, where N is equal to any non-negative integer (e.g. 1.5, 2.5, 3.5 . . . ). Additionally, the diameter of any ultrasound transducer may be 0.5 to 100 times the frequency of the wavelength emitted by the ultrasound transducer. Additionally, any aperture opening may be (N+0.5) times the wavelength emitted by the ultrasound transducers, where N is equal to any non-negative integer (e.g. 0.5, 1.5, 2.5 . . . ).

The materials that can be used for the various components of the medical devices 10, 100, 200, 300 (and/or other medical devices disclosed herein) disclosed herein may include those commonly associated with medical devices. For simplicity purposes, the following discussion makes reference to the catheter shaft 16 and other components of applicable the medical device 10. However, this is not intended to limit the devices and methods described herein, as the discussion may be applied to other similar tubular members and/or components of tubular members or devices disclosed herein.

The catheter shaft 16 and/or other components of the medical device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly (alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly (styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 to achieve the same result.

In some embodiments, a degree of Magnetic Resonance Imaging (MRI) compatibility is imparted into the medical device 10. For example, the medical device 10 or portions thereof, may be made of a material that does not substantially distort the image and create substantial artifacts (e.g., gaps in the image). Certain ferromagnetic materials, for example, may not be suitable because they may create artifacts in an MRI image. The medical device 10 or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An ultrasound medical device, comprising:
an elongate shaft having a distal end region;
a housing coupled to the elongate shaft;
a first ultrasound transducer coupled to the housing; and
a second ultrasound transducer coupled to the housing;
wherein the first ultrasound transducer is configured to generate a first ultrasound wave;
wherein the second ultrasound transducer is configured to generate a second ultrasound wave;
and wherein the first ultrasound wave constructively interferes with the second ultrasound wave to generate a localized pressure wave in order to transmit energy to a target region.

2. The ultrasound device of claim 1, wherein the first ultrasound transducer and the second ultrasound transducer are positioned within the housing.

3. The ultrasound device of claim 2, wherein the housing includes a first housing member and a second housing member, and wherein the first housing member is configured to attach to the second housing member in a clamshell configuration.

4. The ultrasound device of claim 3, wherein the housing member includes a first aperture, and wherein the aperture is positioned between the first ultrasound transducer and the second ultrasound transducer.

5. The ultrasound device of claim 4, wherein the transmitted energy is configured to pass through the aperture.

6. The ultrasound medical device of claim 1, further comprising a third ultrasound transducer and a fourth ultrasound transducer, and wherein the first ultrasound transducer and the second ultrasound transducer form a first pair of ultrasound transducers, wherein the third ultrasound transducer and the fourth ultrasound transducer form a second pair of ultrasound transducers.

7. The ultrasound medical device of claim 6, wherein the first pair of ultrasound transducers are longitudinally aligned with the second pair of ultrasound transducers.

8. The ultrasound medical device of claim 1, further comprising a balloon coupled to the elongate shaft, and wherein the balloon is designed to shift between a first unexpanded configuration and an expanded configuration wherein the balloon is in contact with the target region, and wherein the housing is positioned underneath the balloon.

9. The ultrasound medical device of claim 8, wherein the balloon is configured to be expanded by an inflation media, and wherein the generation of the pressure wave further includes the generation of a bubble within the inflation media.

10. The ultrasound medical device of claim 8, wherein the pressure wave is generated within the balloon.

11. The ultrasound medical device of claim 8, further comprising a third ultrasound transducer and a fourth ultrasound transducer positioned in the housing, and wherein the first ultrasound transducer and the second ultrasound transducer form a first pair of ultrasound transducers, wherein the third ultrasound transducer and the fourth ultrasound transducer form a second pair of ultrasound transducers.

12. The ultrasound device of claim 11, wherein the first pair of ultrasound transducers are configured to generate a first pressure wave within the balloon, and wherein the second pair of transducers are configured to generate a second pressure wave within the balloon, and wherein the generation of the first pressure wave occurs substantially simultaneously with generation of the second pressure wave.

13. The ultrasound medical device of claim 11, wherein the first pair of ultrasound transducers are configured to generate a first cavitation event within the balloon, and wherein the second pair of transducers are configured to generate a second cavitation event within the balloon, and wherein the first cavitation event occurs substantially simultaneously with the second cavitation event.

14. The ultrasound medical device of claim 1, wherein the first ultrasound transducer, the second ultrasound transducer or both the first and the second ultrasound transducers are designed to transmit an ultrasound wavelength at a first frequency, and wherein the first ultrasound transducer is longitudinally spaced away from the second ultrasound transducer between a distance of 0.5 to 3.5 times the first frequency wavelength.

15. An ultrasound system, comprising:
a generator;
an elongate shaft having a distal end region and a proximal region coupled to the generator;
a balloon coupled to the distal end region of the elongate shaft; and
a set of ultrasound transducers coupled to the generator and positioned under the balloon;
wherein the set of ultrasound transducers are configured to generate a first ultrasound wave and a second ultrasound wave, and wherein the first ultrasound wave constructively interferes with the second ultrasound wave to generate a localized cavitation event within the balloon in order to transmit energy to a target region.

16. The ultrasound system of claim 15, wherein the cavitation event further includes the generation of a pressure wave within the balloon.

17. A method for treating a blood vessel, the method comprising:
disposing a medical device within the blood vessel at a position adjacent to a lesion, the medical device comprising:
an elongate shaft having a distal end region;
a balloon coupled to the distal end region;
a first ultrasound transducer coupled to the shaft and positioned under the balloon; and
a second ultrasound transducer coupled to the shaft and positioned under the balloon;
generating a first ultrasound wave with the first ultrasound transducer; and
generating a second ultrasound wave with the second ultrasound transducer, wherein the first ultrasound wave constructively interferes with the second ultrasound wave to generate a force to at least partially break apart the lesion.

18. The method of claim 17, wherein the balloon is designed to shift between a first unexpanded configuration and an expanded configuration wherein the balloon is in contact with the lesion.

19. The method of claim 17, wherein the balloon is configured to be expanded by an inflation media, and wherein the generation of the force further includes the generation of a bubble within the inflation media.

20. The method of claim 17, wherein the generation of the force further includes the generation of a pressure wave within the balloon.
